(19) United States
(12) Patent Application Publication
(10) Pub. No.: US 2021/0322887 A1
(43) Pub. Date: Oct. 21, 2021

(54) SYSTEMS AND METHODS FOR ADAPTING USER EXPERIENCE IN A DIGITAL EXPERIENCE BASED ON PSYCHOLOGICAL ATTRIBUTES OF INDIVIDUAL USERS

(71) Applicant: 12traits, Inc., Manhattan Beach, CA (US)

(72) Inventor: Joseph Jack Schaeppi, Maple Grove, MN (US)

(21) Appl. No.: 16/854,660

(22) Filed: Apr. 21, 2020

(51) Int. Cl. A63F 13/795; G06N 5/04; A61B 5/16; G06K 9/62

(52) U.S. Cl. CPC A63F 13/795; G06N 5/04; A63F 2300/5566; G06K 9/6223; A61B 5/16

(57) ABSTRACT

Systems and methods for adapting user experience in a digital experience based on psychological attributes of individual users are disclosed. Exemplary implementations may: store, in electronic storage, information associated with the individual users; execute an instance of the digital experience and to use the instance to generate game information that is transmitted to client computing platforms; obtain stated information provided by the users; determine, based on the sets of answers, sets of psychological parameter values for the individual users; identify, based on the sets of psychological parameter values for the individual users, clusters of users that have similar sets of psychological parameter values; group the users into cohorts that correspond to the clusters; and adapt the digital experience for the individual users based on their cohorts.

### FIELD OF THE DISCLOSURE

The present disclosure relates to systems and methods for adapting user experience in a digital experience based on psychological attributes of individual users.

### BACKGROUND

It is known that within digital environments, users may be “classified” based on behaviors within the digital environments. For example, within online games, users tend to participate in various aspects of the game and eschew others. These aspects may include Player versus Player (“PvP”) gameplay in which users seek to engage in competing directly with (e.g., battling) other players within the game and Player versus Environment (“PvE”) gameplay in which users seek to complete “quests” or other tasks within the game to gain points, virtual items, and/or other rewards. For example, within websites and/or applications, users may spend more or less time on particular pages of a website and/or application, purchase from a particular category and not others, and click on particular information, among others.

Typically, users of a digital environment are not “classified” (e.g., into a player type) until after they have begun interacting with the digital environment and have a large enough sample of use for classifications and/or predictions about future activities to be made based on behaviors within the environment. Users sometimes have difficulty “finding” the activities and/or features on a digital environment that will make them long term, highly engaged users. Even upon classification based on the behaviors, such classifications may not remain accurate over time. Further, such techniques fail to understand users psychologically as they interact within a digital environment or experience that may consequently allow the experience to be uniquely adapted to a particular user.

### SUMMARY

One aspect of the present disclosure relates to creating user experiences that contribute to user engagement of a digital experience. Upon initiating the digital experience, users may be presented a psychological assessment. Based on answers provided by the users, psychological attributes may be determined for the individual users. The users may be classified into groups of users with similar psychological attributes unique to the particular digital experience. Groups that the users are classified into may be distinct for individual digital experiences (e.g., an online game, website, mobile application, etc.). Subsequently, the individual groups of users may be associated with groups within the digital experience. Adaptations to the digital experience may be made for users of the groups within the digital experience based on the groups within the digital experience.

One aspect of the present disclosure relates to a system configured for adapting user experience in the digital experience based on psychological attributes of individual users. The system may include one or more hardware processors configured by machine-readable instructions and electronic storage. The machine-readable instructions may include one or more instruction components. The instruction components may include one or more of game component, information component, group component, game adaptation component, and/or other instruction components.

The game component may be configured to execute an instance of the digital experience and to use the instance to generate game information that is transmitted to client computing platforms. The game information may facilitate presentation of the digital experience to the users via the client computing platforms. The client computing platforms may be associated with the users.

The information component may be configured to obtain stated information provided by the users. The stated information may include sets of answers to questions that relate to psychological attributes. The individual sets of answers may be provided by individual ones of the users. In some implementations, the sets of answers may include a first set of answers provided by a first user. In some implementations, the electronic storage may be configured to store, in electronic storage, information such as the stated information associated with the individual users. The information component may be configured to determine, based on the sets of answers, sets of psychological parameter values for the individual users. In some implementations, a first set of psychological parameter values may be determined for the first user.

The group component may be configured to identify clusters of users that have similar sets of psychological parameter values. The identification of clusters may be based on the sets of psychological parameter values for the individual users. In some implementations, the clusters may include a first cluster that includes the first user on the basis of the first set of psychological parameter values. The group component may be configured to group the users into cohorts that correspond to the clusters. In some implementations, the cohorts may include a first cohort that corresponds to the first cluster. The first user may be grouped into the first cohort by virtue of being included in the first cluster.

The game adaptation component may be configured to adapt the digital experience for the individual users based on their cohorts. In some implementations, a first adaptation to the digital experience may be made for the first cohort of users, including the first user.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured for adapting user experience in a digital experience based on personality of individual users, in accordance with one or more implementations.

FIG. 2 illustrates a method for adapting user experience in a digital experience based on personality of individual users, in accordance with one or more implementations.

FIG. 3A illustrates an example implementation for adapting user experience in a digital experience based on personality of individual users, in accordance with one or more implementations.

FIG. 3B illustrates an example implementation for adapting user experience in a digital experience based on personality of individual users, in accordance with one or more implementations.

### DETAILED DESCRIPTION

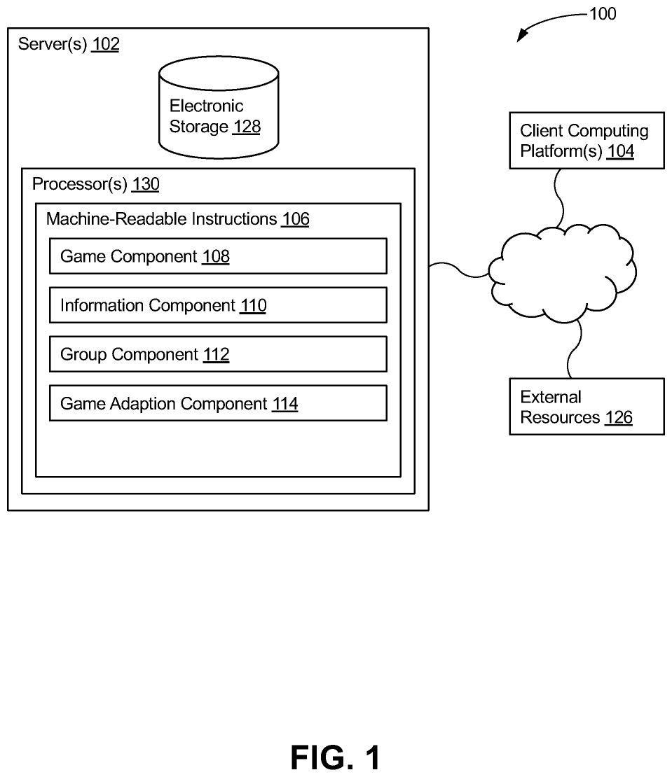

FIG. 1 illustrates a system 100 configured for adapting user experience in a digital experience based on psychological attributes of individual users, in accordance with one or more implementations. In some implementations, system 100 may include one or more servers 102. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via client computing platform(s) 104.

Server(s) 102 may be configured by machine-readable instructions 106. Machine-readable instructions 106 may include one or more instruction components. The instruction components may include computer program components. The instruction components may include one or more of game component 108, information component 110, group component 112, game adaptation component 114, and/or other instruction components.

Game component 108 may be configured to execute an instance of the digital experience and to use the instance to generate game information that is transmitted to client computing platforms. The digital experience may include one or more of an online game, desktop game, mobile game, web application, website, mobile application, and/or other digital experiences. The game information may facilitate presentation of the digital experience to the users via the client computing platforms. The client computing platforms may be associated with the users.

The digital experience, such as an online game, may be provided via a virtual space, and may include a plurality of resource types and/or maps. An instance of the virtual space may be executed by computer components to determine views of the virtual space. The views may then be communicated (e.g., via streaming, via object/position data, and/or other information) from server(s) 102 and/or sources to client computing platforms 104 for presentation to users. The view determined and transmitted to a given client computing platform 104 may correspond to a location in the virtual space (e.g., the location from which the view is taken, the location the view depicts, and/or other locations), a zoom ratio, a dimensionality of objects, a point-of-view, and/or view parameters. One or more of the view parameters may be selectable by the user.

The instance of the virtual space may comprise a simulated space that is accessible by users via clients (e.g., client computing platform(s) 104) that present the views of the virtual space to a user. The simulated space may have a topography, express ongoing real-time interaction by one or more users, and/or include one or more objects positioned within the topography that are capable of locomotion within the topography. In some instances, the topography may be a 2-dimensional topography. In other instances, the topography may be a 3-dimensional topography. The topography may include dimensions of the space, and/or surface features of a surface or objects that are “native” to the space. In some instances, the topography may describe a surface (e.g., a ground surface) that runs through at least a substantial section of the space. In some instances, the topography may describe a volume with one or more bodies positioned therein (e.g., a simulation of gravity-deprived space with one or more celestial bodies positioned therein). The instance executed by the computer components may be synchronous, asynchronous, and/or semi-synchronous.

The above description of the manner in which views of the virtual space are provided is not intended to be limiting. The virtual space may be expressed in a more limited, or richer, manner. For example, views determined for the virtual space may be selected from a limited set of graphics depicting an event in a given place within the virtual space. The views may include additional content (e.g., text, audio, pre-stored video content, and/or other content) that describes particulars of the current state of the place, beyond the relatively generic graphics. For example, a view may include a generic battle graphic with a textual description of the opponents to be confronted. Other expressions of individual places within the virtual space are contemplated.

Within the instance(s) of the virtual space, users may control characters, objects, simulated physical phenomena (e.g., wind, rain, earthquakes, and/or other phenomena), and/or other elements within the virtual space to interact with the virtual space and/or each other. The user characters may include avatars. As used herein, the term “user character” may refer to an object (or group of objects) present in the virtual space that corresponds to an individual user. The user character may be controlled by the user with which it is associated.

User-controlled element(s) may move through and interact with the virtual space (e.g., non-user characters in the virtual space, other objects in the virtual space). The user-controlled elements controlled by and/or associated with a given user may be created and/or customized by the given user. The user may have an “inventory” of virtual goods and/or currency (e.g., resources of the plurality of resource types) that the user can use (e.g., by manipulation of a user character or other user-controlled element, and/or other items) to perform (in-game) actions within the virtual space.

The users may participate in the instance of the virtual space by controlling one or more of the available user-controlled elements in the virtual space. Control may be exercised through control inputs and/or commands input by the users through client computing platforms 104. The users may interact with each other through communications exchanged within the virtual space. Such communications may include one or more of textual chat, instant messages, private messages, voice communications, and/or other communications. Communications may be received and entered by the users via their respective client computing platforms 104. Communications may be routed to and from the appropriate users through server(s) 102. In implementations, the game component 108 may also be configured to implement the customizations determined for the individual users.

Information component 110 may be configured to obtain stated information provided by the users. The stated information may include sets of answers to questions that relate to psychological attributes. The individual sets of answers may be provided by individual ones of the users. In some implementations, game component 108 may be configured to effectuate, via graphical user interfaces of the client computing platforms 104, presentation of the questions. The sets of answers may be transmitted via a network to the one or more processors (i.e., information component 110). In some implementations, the sets of answers may include a first set of answers provided by a first user.

In some implementations, information component 110 may be configured to obtain the stated information that includes a second set of answers provided by a second user. Thus, the set of answers may include the first set of answers and the second set of answers.

Information component 110 may be configured to determine sets of psychological parameter values for the individual users. The determination of the sets of psychological parameter values may be based on the sets of answers provided by the individual users. As a result, a first set of psychological parameter values may be determined for the first user and a second set of psychological parameter values for the second user may be determined. The first set may be based on the first set of answers provided by the first user. The second set may be based on the second set of answers provided by the second user.

The psychological parameter values may characterize, by way of non-limiting example, achievement motivation, motivation, personality inventory, cultural values, competitiveness, positive and negative affect before, during, and/or after engagement with the digital experience (i.e., emotions), communication style, personal values, daily routines/activities, life/gaming pain points, life/gaming hopes and aspirations, wellbeing, user experience, gaming/experience using time, subscription behavior, affinity information, personality, emotional style, goal orientation, goal commitment, ego and task orientation, relatedness, sense of community, social influence, social identity, group identification, we-identity, quality of life, satisfaction with life, work-related quality of life, mindfulness, happiness, emotional intelligence, self-awareness/internal awareness, external awareness, connectedness to nature, social connectedness, social bonding, perceived stress, depression, anxiety, decision-making style, thinking style, critical thinking, cognitive approach to learning, learning style, attributional style, internality-externality, stability-instability, global-specific, creativity, curiosity, playfulness, exploration, mental strength, grit, flourishing, gratitude, inspiration, spirituality, hedonism, materialism/material values, perceptions, sentiments, and/or other psychological parameters.

Achievement motivation may include compensatory effort, competitiveness, confidence in success, dominance, eagerness to learn, engagement, fearlessness, flexibility, flow, goal setting, independence, internality, persistence, preference in difficult tasks, pride in productivity, self-control, status orientation, ambition, self-assurance, and/or other psychological parameters. Motivation may include mastery, purpose, autonomy, and/or other psychological parameters.

Personality inventory may include neuroticism, openness, conscientiousness, extraversion, and agreeableness and/or other psychological parameters. Neuroticism may include anxiety, impulsiveness, vulnerability, and/or other psychological parameters. Openness may include fantasy, feelings/empathy, action, and/or other psychological parameters. Conscientiousness may include achievement striving, competence, self-discipline, and/or other psychological parameters. Extraversion may include warmth assertiveness, activity, and/or other psychological parameters. Agreeableness may include trust, altruism, modesty, and/or other psychological parameters.

Cultural values may include individualism, indulgence, long term orientation, masculinity, power distance, uncertainty avoidance, and/or other psychological parameters. Competitiveness may include avoidant, collaborative, competitive affectivity, dependent, dominant, general competitiveness, independent, personal enhancement, and/or other psychological parameters.

Positive and negative affect before, during, and/or after engaging in the digital experience may include hostility, joviality, negative emotions, positive emotions, sadness, self-assurance, and/or other psychological parameters. Communication style may include feeler, intuitor, sensor, thinker, and/or other psychological parameters.

Wellbeing may include social wellbeing, psychological wellbeing, physical wellbeing, physical activity, sleep, bounded reciprocity, resilience grit, and/or other psychological parameters.

Personality may include anger, hostility, depression, self-conscientiousness, excitement-seeking, positive emotions, gregariousness, ideas, values, aesthetics, tender-mindedness, straightforwardness, compliance, deliberation, order, dutifulness, and/or other psychological parameters.

Emotional style may include resilience, outlook, social intuition, self-awareness, sensitivity to context, attention, and/or other psychological parameters.

Goal orientation may include mastery approach/learning goal orientation, performance approach/performance goal orientation, performance avoid/avoidance goal orientation, and/or other psychological parameters.

Work-related quality of life may include structure, boundaries, focus, efficiency, information provision, communication, psychological support, stress at/from work, psychological safety, connectedness with team, motivation to work, adaptability, job/career satisfaction, control at work, home-work interface, general wellbeing, working conditions, and/or other psychological parameters.

Mindfulness may include observing, describing, acting with awareness, non-judgment, non-reactivity, and/or other psychological parameters.

Emotional intelligence may include emotion perception, emotion expression, emotion management, emotion regulation, impulse control, relationships, stress management, and/or other psychological parameters.

Social connectedness may include social connectedness, loneliness, membership self-esteem, private self-esteem, public self-esteem identity self-esteem, interdependent self, independent self, social avoidance, social distress, and/or other psychological parameters.

Decision-making style may include respected, confident, spontaneous, dependent, vigilant, avoidant, brooding, intuitive, anxious, and/or other psychological parameters.

Thinking style may include intuitive, experiential, analytical, rational, and/or other psychological parameters.

Cognitive approaches to learning may include avoidant, participative, competitive, collaborative, dependent, independent, and/or other psychological parameters.

Learning style may include visual (spatial), aural (auditory-musical), verbal (linguistic), physical (kinesthetic), logical (mathematical), social (interpersonal), solitary (intrapersonal), and/or other psychological parameters.

Mental strength may include tenacity, confidence, optimism, adaptability, self-awareness, reliability, responsibility, well-being, and/or other psychological parameters.

Flourishing may include positive emotion, engagement, relationships, meaning, accomplishment, health, loneliness, and/or other psychological parameters.

By way of non-limiting example, the psychological parameter values of the psychological parameters may be a number score on a predetermined range unique to each psychological parameter, a letter score, and/or other type of value than may characterize a particular user as whole.

In some implementations, information component 110 may be configured to store, in electronic storage 128, information (e.g., the sets of answers, the psychological parameter values) associated with the individual users. In some implementations, the determined psychological parameter values for individual users may be communicated with entities (e.g., game companies, website developers, user experience companies, etc.). The entities may download, export, purchase, subscribe to, obtain in real-time, and/or other obtainments the determined psychological parameter values for individual users.

Group component 112 may be configured to identify clusters of users that have similar sets of psychological parameter values. The identification of clusters may be based on the sets of psychological parameter values for the individual users. The identifying of the clusters of the users may include, by way of non-limiting example, latent class analysis, hierarchical clustering, k-means clustering, mean-shifting clustering, machine learning, dimensionality reduction, principle component analysis, supervised learning, and/or other grouping techniques. The users may be classified into groups of users with similar sets of psychological parameter values unique to a particular digital experience. Groups that the users are grouped into may be distinct for individual digital experiences (e.g., an online game, website, mobile application, etc.). The clusters may include a first cluster and a second cluster. The first cluster may include the first user on the basis of the first set of psychological parameter values. The second cluster may include other users that have other similar sets of psychological parameter values. The second cluster may include the second user on the basis of the second set of psychological parameter values.

Group component 112 may be configured to group the users into cohorts that correspond to the clusters. The cohorts may be groups within the digital experience digital experience that are grouped with regard to the digital experience. By way of non-limiting example, the cohorts may be grouped based on species of virtual characters. The cohorts may include a first cohort that corresponds to the first cluster and a second cohort that corresponds to the second cluster. The first user may be grouped into the first cohort by virtue of being included in the first cluster. The other users may be grouped into the second cohort by virtue of being included in the second cluster. The second user may be grouped into the second cohort by virtue of being included in the second cluster.

Game adaptation component 114 may be configured to adapt the digital experience for the individual users based on their cohorts. As a result, a first adaptation to the digital experience may be made for the first cohort of users, including the first user. A second adaptation to the digital experience may be made for the second cohort of users, including the second user. The adaptations to the digital experience may be modifications to user experiences for the users of the individual cohorts. Modifications to the user experiences may result in a plurality of variations of a digital experience. For example, based on the cohort that the first user is included in, the user experience may be oriented towards a storytelling experience versus a competition experience. For example, based on the determined psychological parameter values of a particular user, the user experience (e.g., storyline path, encountered opponents, virtual content, etc.) may be modified to be unique to the particular user. The adaptations to the digital experience may be modifications to the appearance and/or aesthetic of the digital experience. For example, based on the cohort that the second user is included in, the theme (e.g., brightness, colors, size, etc.) and/or time of day (e.g., lighting of background) of the digital experience may be modified. The adaptations may be are designed to enhance prospective usage of the digital experience by the users.

By way of non-limiting example, the adaptations may include one or more of a recommendation, a suggestion, an adjusted difficulty setting, a piece of game content made available, an omission of game content, means of communication, a communication, an offer to sell one or more virtual items made available, an adjustment of an offer to sell one or more virtual items, an offer to sell bundles or packages of virtual items, development of skills/abilities at a slower or faster rates, means of obtainment of game content, user matching, toxic user detection, and/or other adaptations.

Adapting the recommendations may include adjusting a particular selection and/or action, determined based on their cohort, that the user is advised to select and/or do. Adapting the suggestions may include adjusting particular ideas, plans, and/or strategies for the user to consider executing, following, and/or is determined they will enjoy. The adjusted difficulty setting may adjust how challenging one or more aspects of the digital experience are. The suggestions and/or recommendations may be adapted such that non-player characters may provide different guidance based on the cluster and/or the cohort and/or Pop-ups directing the user to content can be adapted based on based on the cluster and/or the cohort.

By way of non-limiting example, adjusting difficulty settings may include adjusting how challenging the digital experience (e.g., online game) as a whole is, how challenging particular tasks are (e.g., building, battling, problem solving, etc.), how challenging it is to complete one or more levels, adjusting how challenging it is to complete a level with every advancement of a level, and/or other difficulty setting adjustments.

Game content made available or omitted by adaptations may include game content that is provided to a user when performing actions within the digital experience such as exploring a map, researching a technology or skill, purchasing an item within the digital experience, completing an achievement within the digital experience, and/or other information. Such game content (or pieces thereof) may include one or more of a player-controlled character, a non-player-controlled character, a task, a quest, an assignment, a mission, a level, a chapter, a mini-game, a virtual item, a virtual resource (e.g., weapon, tool), of in-game powers, in-game skills, in-game technologies, and/or other pieces of game content. By way of non-limiting example, virtual items may include one or more of clothing, pets, transportation units (e.g., aircrafts, motor vehicles, watercrafts, etc.), units, buildings, and/or other virtual items.

Adapting the means of communication may include adjusting the means of communication made available to be used contemporaneously, made available to be used one at a time, omitted, and/or other adjustments on means of communication. The means of communication may include one or more of a textual chat, instant messages, private messages, voice communications, video communications, in-game communication, out-of-game communication, hybrid communication (i.e., in-game and out-of-game), and/or other means of communication. Adapting the communication may include adjusting when communications (e.g., messages, calls) are sent, when communications are received, to whom communications are sent, from whom communications are received, and/or other adjustments on communications.

Adjusting the offer to sell one or more virtual items made available may adjust the price (e.g., money, coins, tokens, stars, etc.) the users must pay for the one or more virtual items or other game content. Adjusting the offer to sell one or more virtual items may adjust which virtual items or game content may be offered to the users to purchase. The offer to sell bundles or packages of virtual items may include adjusting the bundles available for purchase in an online store for the game, and/or a bundle being provided to the user at the beginning of a digital experience. These adjustments may also include the items included together, the prices of items and other related characteristics.

The means of obtainment of the game content may include by discovery (e.g., exploring a map, in-game research of techs or skills only available to certain users, and/or other discovery), unlocking (e.g., through purchase), achievement (e.g., reward for a quest, an item drop for a boss kill, other PvE activities), and/or other means of obtainment.

Adapting user matching may include more accurately matching users for social circumstances, for competition circumstances, and/or other circumstances. Adapting user matching for competition circumstances may be further adapted for various competition styles. Competition styles may include individual racing, group efforts, group racing, friendly competitions, high intensity competitions, and/or other competition styles.

Adapting toxic user detection may include adapting which individual users or types of users based on their corresponding group and/or cohort may be detected and indicated as toxic (e.g., offensive, harmful, dangerous) to a particular user and/or the digital experience as a whole.

Adapting the digital experience may include one or more modifications to the appearance, aesthetic, and/or other user interface presentations of the digital experience. By way of non-limiting example, user interface presentations may include one or more of colors, layout, landscape, animations, fonts, font sizes, shapes, user interface elements, and/or other user interface presentations. User interface elements may be configured to facilitate user interaction with a user interface, user entry, and/or selection. By way of non-limiting illustration, the user interface elements may include one or more of text input fields, drop down menus, check boxes, display windows, virtual buttons, and/or other user interface elements.

In some implementations, adaptations to the digital experience may be made for individual users without the individual users being grouped into a cluster and/or cohort. Thus, such adaptations may be unique to the particular user.

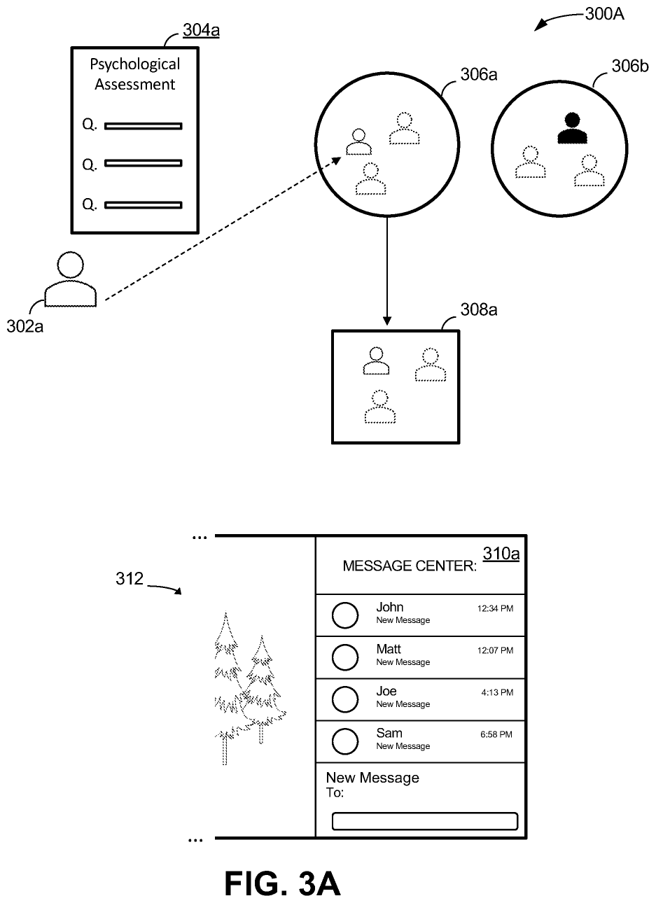

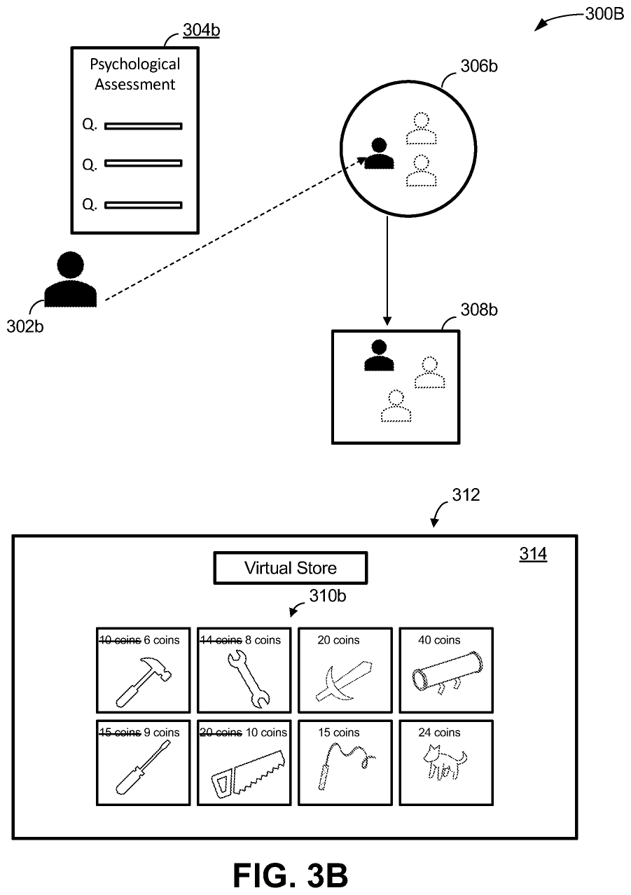

FIG. 3A illustrates an example implementation for adapting user experience in an online game based on psychological attributes of individual users, in accordance with one or more implementations. FIG. 3A illustrates adaptation implementation 300A. Adaptation implementation 300A includes a question set 304a that may be presented to a user 302a. Answers provided by user 302a may be determinative of psychological parameter values. Based on the psychological parameter values, user 302a may be included in a cluster 306a of users with similar psychological parameter values instead of a cluster 306b. Cluster 306a may correspond to a cohort 308a of a digital experience 312. Based on the similar psychological parameter values of cohort 308a (and/or cluster 306a), it may be determined that the users, including user 302a, of cluster 306a prefer message communication over voice or video communication. Thus, digital experience 312 may be adapted to include adaptation 310a so that a Message Center is always present on a graphical user interface of a client computing platform of user 302a.

FIG. 3B illustrates an example implementation for adapting user experience in a digital experience based on psychological attributes of individual users, in accordance with one or more implementations. FIG. 3B illustrates adaptation implementation 300B. Adaptation implementation 300B includes a question set 304b that may be presented to a user 302b. Question set 304b may be the same as or similar to question set 302a presented to user 302a of FIG. 3A. Similar to the answers provided by user 302a of FIG. 3A, answers provided by user 302b may be determinative of psychological parameter values. Based on the psychological parameter values, user 302b may be included in cluster 306b of users with similar psychological parameter values instead of cluster 306a with user 302a. Cluster 306b may correspond to a cohort 308b of digital experience 312. Based on the similar psychological parameter values of cohort 308b (and/or cluster 306b), it may be determined that the users, including user 302b, of cluster 306b are creative and enjoy building. Thus, a virtual store 314 of digital experience 312 may be adapted to include adaptation 310b so that tools are offered at a discount for user 302b to purchase and weapons or pets are not offered at a discount.

In some implementations, information component 110 may be configured to determine behavioral information of the users in the clusters. The behavioral information may include behaviors of the users. By way of non-limiting example, the behaviors may include one or more of actions of the users, purchases of the users, communication of the users with particular users, completion of tasks by the users, uncompletion of tasks by the users, failure of tasks by the users, frequent interaction with content of the users, formation of alliances by the users and the types of the alliances, and/or other behaviors. In some implementations, the determined behavioral information may include at least two of the behaviors of which the adapting of the digital experience (i.e., the adaptations) is based on. In some implementations, the determined behavioral information of the clusters may be stored to electronic storage 128. In some implementations, the grouping of the users, by group component 112, into the cohorts that correspond to the clusters may be based on the behavioral information.

In some implementations, server(s) 102, client computing platform(s) 104, and/or external resources 126 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 102, client computing platform(s) 104, and/or external resources 126 may be operatively linked via some other communication media.

A given client computing platform 104 may include one or more processors configured to execute computer program components. The computer program components may be configured to enable an expert or user associated with the given client computing platform 104 to interface with system 100 and/or external resources 126, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 126 may include sources of information outside of system 100, external entities participating with system 100, and/or other resources. In some implementations, some or all of the functionality attributed herein to external resources 126 may be provided by resources included in system 100.

Server(s) 102 may include electronic storage 128, one or more processors 130, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 128 may comprise non-transitory storage media that electronically stores information. The electronic storage media of electronic storage 128 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 128 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 128 may include one or more virtual storage resources (e.g., cloud storage, a virtual private network, and/or other virtual storage resources). Electronic storage 128 may store software algorithms, information determined by processor(s) 130, information received from server(s) 102, information received from client computing platform(s) 104, and/or other information that enables server(s) 102 to function as described herein.

Processor(s) 130 may be configured to provide information processing capabilities in server(s) 102. As such, processor(s) 130 may include one or more of a digital processor, an analog processor, a digital circuit designed to process information, an analog circuit designed to process information, a state machine, and/or other mechanisms for electronically processing information. Although processor(s) 130 is shown in FIG. 1 as a single entity, this is for illustrative purposes only. In some implementations, processor(s) 130 may include a plurality of processing units. These processing units may be physically located within the same device, or processor(s) 130 may represent processing functionality of a plurality of devices operating in coordination. Processor(s) 130 may be configured to execute components 108, 110, 112, and/or 114, and/or other components. Processor(s) 130 may be configured to execute components 108, 110, 112, and/or 114, and/or other components by software; hardware; firmware; some combination of software, hardware, and/or firmware; and/or other mechanisms for configuring processing capabilities on processor(s) 130. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, and/or 114 are illustrated in FIG. 1 as being implemented within a single processing unit, in implementations in which processor(s) 130 includes multiple processing units, one or more of components 108, 110, 112, and/or 114 may be implemented remotely from the other components. The description of the functionality provided by the different components 108, 110, 112, and/or 114 described below is for illustrative purposes, and is not intended to be limiting, as any of components 108, 110, 112, and/or 114 may provide more or less functionality than is described. For example, one or more of components 108, 110, 112, and/or 114 may be eliminated, and some or all of its functionality may be provided by other ones of components 108, 110, 112, and/or 114. As another example, processor(s) 130 may be configured to execute one or more additional components that may perform some or all of the functionality attributed below to one of components 108, 110, 112, and/or 114.

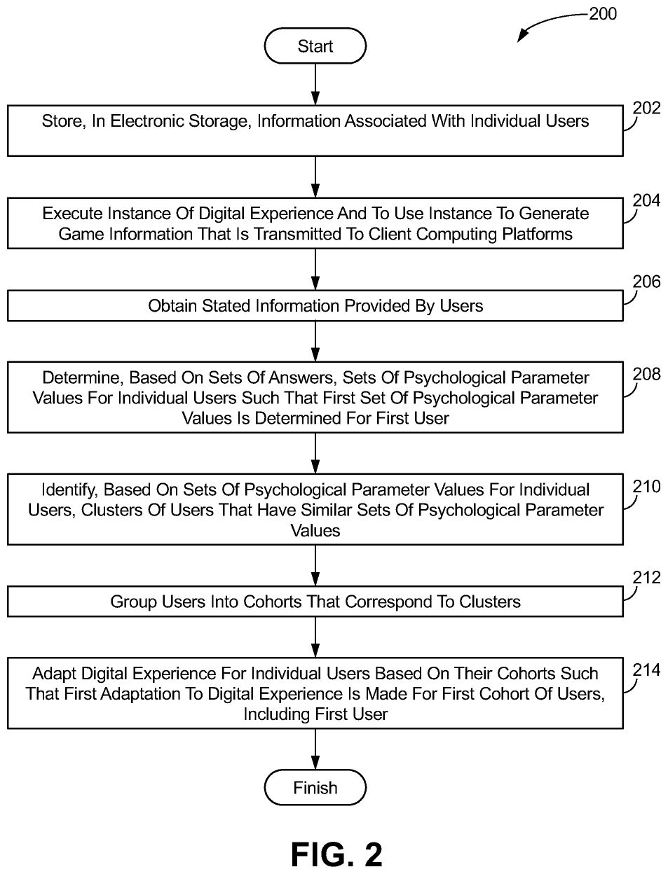

FIG. 2 illustrates a method 200 for adapting user experience in a digital experience based on psychological attributes of individual users, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include storing, in electronic storage, information associated with the individual users. Operation 202 may be performed by one or more hardware processors configured by machine-readable instructions including a component that is the same as or similar to information component 110 and electronic storage 128, in accordance with one or more implementations.

An operation 204 may include executing an instance of the digital experience and to use the instance to generate game information that is transmitted to client computing platforms. The game information may facilitate presentation of the digital experience to the users via the client computing platforms. The client computing platforms may be associated with the users. Operation 204 may be performed by one or more hardware processors configured by machine-readable instructions including a component that is the same as or similar to game component 108, in accordance with one or more implementations.

An operation 206 may include obtaining stated information provided by the users. The stated information may include sets of answers to questions that relate to psychological attributes. The individual sets of answers may be provided by individual ones of the users such that the sets of answers include a first set of answers provided by a first user. Operation 206 may be performed by one or more hardware processors configured by machine-readable instructions including a component that is the same as or similar to information component 110, in accordance with one or more implementations.

An operation 208 may include determining, based on the sets of answers, sets of psychological parameter values for the individual users such that a first set of psychological parameter values is determined for the first user. Operation 208 may be performed by one or more hardware processors configured by machine-readable instructions including a component that is the same as or similar to information component 110, in accordance with one or more implementations.

An operation 210 may include identifying, based on the sets of psychological parameter values for the individual users, clusters of users that have similar sets of psychological parameter values. The clusters may include a first cluster that includes the first user on the basis of the first set of psychological parameter values. Operation 210 may be performed by one or more hardware processors configured by machine-readable instructions including a component that is the same as or similar to group component 112, in accordance with one or more implementations.

An operation 212 may include grouping the users into cohorts that correspond to the clusters. The cohorts may include a first cohort that corresponds to the first cluster. The first user may be grouped into the first cohort by virtue of being included in the first cluster. Operation 212 may be performed by one or more hardware processors configured by machine-readable instructions including a component that is the same as or similar to group component 112, in accordance with one or more implementations.

An operation 214 may include adapting the digital experience for the individual users based on their cohorts such that a first adaptation to the digital experience is made for the first cohort of users, including the first user. Operation 214 may be performed by one or more hardware processors configured by machine-readable instructions including a component that is the same as or similar to game adaptation component 114, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A system configured to adapt user experience in a digital experience based on psychological attributes of individual users, the system comprising:
electronic storage configured to store information associated with the individual users;
one or more processors configured by machine-readable instructions to:
execute an instance of the digital experience of a digital environment and to use the instance to generate game information that is transmitted to client computing platforms, the game information facilitating presentation of the digital experience to the users via the client computing platforms, wherein the client computing platforms are associated with the users;
obtain, via a network, stated information provided by the users, wherein the stated information includes sets of answers to questions that relate to psychological attributes, wherein the individual sets of answers are provided by individual ones of the users such that the sets of answers include a first set of answers provided by a first user;
determine, based on the sets of answers, sets of psychological parameter values for the individual users such that a first set of psychological parameter values is determined for the first user;
identify, based on the sets of psychological parameter values for the individual users, clusters of users that have similar sets of psychological parameter values, the clusters including a first cluster that includes the first user on the basis of the first set of psychological parameter values;
group the users into cohorts that correspond to the clusters, the cohorts including a first cohort that corresponds to the first cluster, the first user being grouped into the first cohort by virtue of being included in the first cluster; and
adapt the digital experience for the individual users based on their cohorts such that a first adaptation to the digital experience is made for the first cohort of users, including the first user.

2. The system of claim 1, wherein the first adaptation includes one or more of a recommendation, a suggestion, an adjusted difficulty setting, a piece of game content made available, an omission of game content, means of communication, a communication, an offer to sell one or more virtual items made available, an adjustment of an offer to sell one or more virtual items, matching users for particular circumstances, detecting toxic users, and/or adjusted presentation of a user interface.

3. The system of claim 1, wherein the psychological parameter values characterize motivations, emotions, emotional intelligence, cultural values, personal values, communication style, personality, psychological wellbeing, and/or learning style.

4. The system of claim 1, wherein identifying the clusters of the users includes latent class analysis, hierarchical clustering, K-means clustering, mean-shifting clustering, machine learning, dimensionality reduction, and/or principle component analysis.

5. The system of claim 1, wherein the one or more processors are further configured by machine-readable instructions to:
effectuate, via user interfaces of the client computing platforms, presentation of the questions, wherein the sets of answers are transmitted via a network to the one or more processors.

6. The system of claim 1, wherein the one or more processors are further configured by machine-readable instructions to:
determine behavioral information of the users in the clusters, wherein the grouping of the users into the cohorts that correspond to the clusters is based on the behavioral information.

7. The system of claim 6, wherein the behavioral information includes behaviors of the users, the behaviors including actions of the users, purchases of the users, communication of the users with particular users, completion of tasks by the users, uncompletion tasks by the users, failure of tasks by the users, frequent interaction with content of the users, and/or formation of alliances by the users and the types of the alliances.

8. The system of claim 7, wherein the determined behavioral information of the clusters is stored to the electronic storage.

9. The system of claim 6, wherein the determined behavioral information includes at least two of the behaviors of which the adapting of the digital experience is based on.

10. The system of claim 1, wherein the one or more processors are further configured by machine-readable instructions to:
obtain the stated information that includes a second set of answers provided by a second user;
determine, based on the second set of answers, a second set of psychological parameter values for the second user;
identify, based on the second set of psychological parameter values, a second cluster of other users that have similar sets of psychological parameter values, the second cluster including the second user on the basis of the second set of psychological parameter values;
group the other users into a second cohort that corresponds to the second cluster, the second user being grouped into the second cohort by virtue of being included in the second cluster; and
adapt the digital experience for the second cohort of users, including the second user, with a second adaptation.

11. A method for adapting user experience in a digital experience based on psychological attributes of individual users, the method comprising:
storing, in electronic storage, information associated with the individual users;
executing an instance of the digital experience of a digital environment and to use the instance to generate game information that is transmitted to client computing platforms, the game information facilitating presentation of the digital experience to the users via the client computing platforms, wherein the client computing platforms are associated with the users;
obtaining, via a network, stated information provided by the users, wherein the stated information includes sets of answers to questions that relate to psychological attributes, wherein the individual sets of answers are provided by individual ones of the users such that the sets of answers include a first set of answers provided by a first user;
determining, based on the sets of answers, sets of psychological parameter values for the individual users such that a first set of psychological parameter values is determined for the first user;
identifying, based on the sets of psychological parameter values for the individual users, clusters of users that have similar sets of psychological parameter values, the clusters including a first cluster that includes the first user on the basis of the first set of psychological parameter values;
grouping the users into cohorts that correspond to the clusters, the cohorts including a first cohort that corresponds to the first cluster, the first user being grouped into the first cohort by virtue of being included in the first cluster; and
adapting the digital experience for the individual users based on their cohorts such that a first adaptation to the digital experience is made for the first cohort of users, including the first user.

12. The method of claim 11, wherein the first adaptation includes one or more of a recommendation, a suggestion, an adjusted difficulty setting, a piece of game content made available, an omission of game content, means of communication, a communication, an offer to sell one or more virtual items made available, an adjustment of an offer to sell one or more virtual items, matching users for particular circumstances, detecting toxic users, and/or adjusted presentation of a user interface.

13. The method of claim 11, wherein the psychological parameter values characterize motivations, emotions, emotional intelligence, cultural values, personal values, communication style, personality, psychological wellbeing, and/or learning style.

14. The method of claim 11, wherein identifying the clusters of the users includes latent class analysis, hierarchical clustering, K-means clustering, mean-shifting clustering, machine learning, dimensionality reduction, and/or principle component analysis.

15. The method of claim 11, further comprising:
effectuating, via user interfaces of the client computing platforms, presentation of the questions, wherein the sets of answers are transmitted via a network to one or more processors.

16. The method of claim 11, further comprising:
determining behavioral information of the users in the clusters, wherein the grouping of the users into the cohorts that correspond to the clusters is based on the behavioral information.

17. The method of claim 16, wherein the behavioral information includes behaviors of the users, the behaviors including actions of the users, purchases of the users, communication of the users with particular users, completion of tasks by the users, uncompletion tasks by the users, failure of tasks by the users, frequent interaction with content of the users, and/or formation of alliances by the users and the types of the alliances.

18. The method of claim 17, wherein the determined behavioral information of the clusters is stored to the electronic storage.

19. The method of claim 16, wherein the determined behavioral information includes at least two of the behaviors of which the adapting of the digital experience is based on.

20. The method of claim 11, further comprising:
obtaining the stated information that includes a second set of answers provided by a second user;
determining, based on the second set of answers, a second set of psychological parameter values for the second user;
identifying, based on the second set of psychological parameter values, a second cluster of other users that have similar sets of psychological parameter values, the second cluster including the second user on the basis of the second set of psychological parameter values;
grouping the other users into a second cohort that corresponds to the second cluster, the second user being grouped into the second cohort by virtue of being included in the second cluster; and
adapting the digital experience for the second cohort of users, including the second user, with a second adaptation.
